(19) United States
(12) Patent Application Publication
(10) Pub. No.: US 2024/0252371 A1
(43) Pub. Date: Aug. 1, 2024

(54) DRESSING KIT FOR EXTERNAL FIXATION AND EXTREMITIES

(71) Applicant: William MONTROSS, Colorado Springs, CO (US)

(72) Inventor: William MONTROSS, Colorado Springs, CO (US)

(21) Appl. No.: 18/427,656

(22) Filed: Jan. 30, 2024

Related U.S. Application Data
(60) Provisional application No. 63/441,951, filed on Jan. 30, 2023.

(51) Int. Cl. A61F 13/82; A61B 17/62; A61F 13/06; A61F 13/10; A61F 13/74

(52) U.S. Cl. CPC A61F 13/82; A61B 17/62; A61F 13/064; A61F 13/104; A61F 13/74

(57) ABSTRACT

Disclosed are devices, systems and/or methods for protecting and healing injuries and/or other wounds of a patient's extremity.

### CROSS-REFERENCE TO RELATED APPLICATIONS

This application claims priority to and benefit thereof from U.S. Provisional Patent Application No. 63/441,951, filed Jan. 30, 2023, entitled “DRESSING KIT FOR EXTERNAL FIXATION,” the disclosure of which is hereby incorporated herein by reference in its entirety.

### TECHNICAL FIELD

The invention relates to improved devices, systems and methods for protecting and healing injuries and/or other wounds of a patient extremity, such as where the extremity is being supported by an external fixation device and/or within an external support structure or frame.

### BACKGROUND OF THE INVENTION

In many cases, an injured leg or other extremity may require external support and/or reinforcement to effectuate healing of the limb, such as by being held in position within a surgical support structure or frame. Unfortunately, the presence of the frame and other support structures (e.g., fixation pins and/or support rods) can render it difficult or impossible to access limb structures which may require cleaning and/or changing of surgical dressings. Failure to clean and/or refresh wound dressings can lead to infection and/or necrosis of wound tissues. Thus, there is a need in the art for improved devices, systems and methods for cleaning and/or changing surgical dressings of an extremity attached to and/or supported by an external fixation device, external support structure and/or frame.

### BRIEF SUMMARY OF THE INVENTION

The present invention includes the realization of a need in the art for improved devices, systems and methods for cleaning and/or changing surgical dressings of an extremity attached to and/or supported by an external fixation device, external support structure and/or frame. In various embodiments, the devices and systems disclosed herein can be shaped to protect the anatomical structure of the extremity with particular focus on the foot and leg. Desirably, the dressing can be modular so it can easily be modified to fit any wire or pin position on the extremities of the human body particularly the foot. The unique shapes will desirably protect anatomic structures such as bony prominences, neuro vascular structures and tendons that have little protection and which can often be injured if inadequate protection is applied to the area.

### BRIEF DESCRIPTION OF THE SEVERAL VIEWS OF THE DRAWINGS

The foregoing and other features and advantages of the present subject matter will become apparent to those skilled in the art to which the present subject matter relates upon reading the following description with reference to the accompanying drawings.

FIG. 1 depicts a top plan view of one exemplary embodiment of a wound dressing component;

FIG. 2 depicts a side view of the wound dressing component of FIG. 1;

FIG. 3 depict a top plan view of another exemplary embodiment of a wound dressing component;

FIG. 4 depicts a side view of the wound dressing component of FIG. 3;

FIG. 5A depicts a top plan view of a securement tab for use with the wound dressing components of FIGS. 1 and 3;

FIG. 5B depicts a perspective view of the securement tab of FIG. 5A;

FIG. 5C depicts a side view of the securement tab of FIG. 5A;

FIG. 6 depicts a plan view of one exemplary kit containing various wound dressing components described herein;

FIG. 7A depicts a bottom planar view of a human skeletal foot;

FIG. 7B depicts the human skeletal foot of FIG. 7A, with the foot depicted secured into a fixation frame;

FIG. 8A depicts a bottom planar view of the human skeletal foot of FIG. 7A with the wound dressing component of FIG. 1 placed thereupon;

FIG. 8B depicts the human skeletal foot of FIG. 8A in a fixation frame;

FIGS. 9A and 9B depict views of the wound dressing component of FIG. 8A wrapped around the foot;

FIGS. 9C and 9D depicts exemplary securement tabs attached to fixation pins;

FIG. 10A depicts a top planar view of the human skeletal foot of FIG. 7A, with the wound dressing component of FIG. 3 placed thereupon;

FIG. 10B depicts an alternative view of the human skeletal foot and wound dressing component;

FIG. 10C depicts the wound dressing component and foot of FIG. 10A, with the central body positioned against the top of the foot, the center tab bent back and in contact with the patient's leg, and the tab sections wrapping around the sides to the patient's ankle;

FIG. 11 depicts the dressing components fully secured to the foot in a desired position and/or orientation;

FIG. 12A depicts a view of additional exemplary embodiments of a wound dressing component;

FIG. 12B depicts one exemplary wound dressing component of FIG. 12A secured to fixation pins via slits in the component;

FIGS. 12C and 12D depict views of another exemplary wound dressing component secured to fixation pins via slits in the component; and

FIG. 12E depicts the dressing component of FIG. 12A fully secured in a desired position and/or orientation.

### DETAILED DESCRIPTION OF THE INVENTION

The present subject matter relates generally to devices for the fixation and support of extremities. In particular, the present subject matter relates to a wound dressing comprising multiple components that can be quickly and easily secured to a foot or other extremity, especially where the foot or other extremity is secured within an external fixation frame and/or other stabilization and/or securement construct. Desirably, the wound dressing can be quickly and easily attached to the foot or other extremity and can similarly be removed and/or replaced without requiring significant adjustment of the frame components and/or unnecessary manipulation of the extremity.

The best mode for carrying out the invention is desirably presented in terms of its preferred embodiment. However, the invention is not limited to the described embodiment(s), and a person skilled in the art will appreciate that many other embodiments of the invention are possible without deviating from the basic concept of the invention and that any such work around will also fall under scope of this invention. It is envisioned that other styles and configurations of the present invention can be easily incorporated into the teachings of the present invention, and only one particular configuration shall be shown and described for purposes of clarity and disclosure and not by way of limitation of scope.

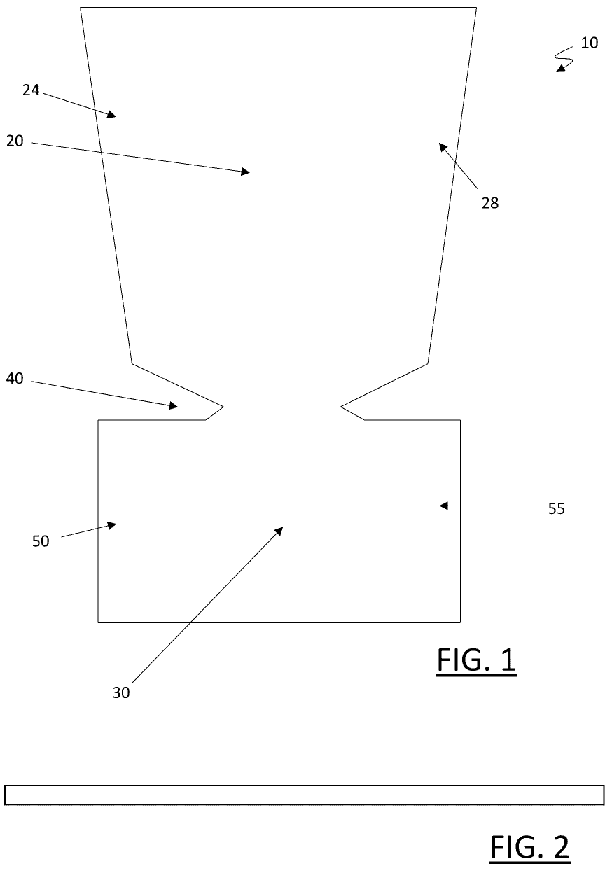

FIGS. 1 and 2 depict views of one exemplary embodiment of a wound dressing component 10, which may comprise a heavy-duty dressing material optionally capable of absorbing fluids, such as an abdominal gauze dressing or ABD pad. The dressing component 10 desirably includes a central body 20 and a base 30, which are connected at a neck section 40. The central body 20 can include body tabs 24 and 28, and the base 30 can include base tabs 50 and 55, with the body tabs desirably decoupled from the base tabs to allow the tabs to wrap around a different sized and/or shaped body part. If desired, the dressing can include features to allow typical 4×4, 2×2, etc., gauze pads to be added for extra protection or absorbency. In various embodiments, a triple thick silver impregnated ABD dressing may be utilized.

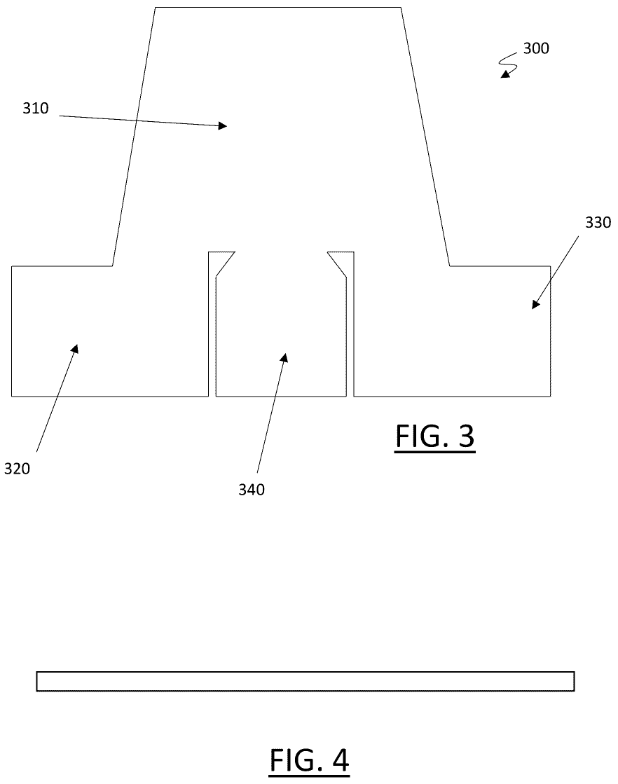

FIGS. 3 and 4 depict views of another exemplary embodiment of a wound dressing component 300, which may comprise a heavy-duty dressing material optionally capable of absorbing fluids, such as an abdominal gauze dressing or ABD pad. The dressing component 300 desirably includes a central section 310, depicted in this embodiment as a generally trapezoidal shaped section, with a pair of tab sections 320 and 330 and a center tab section 340, with the central base 320 connected to the central and tab sections. In this embodiment, the center and tab sections are similarly sized and configured to accommodate a different sized and/or shaped body part, and the dressing can optionally include features to allow typical 4×4, 2×2, etc., gauze pads to be added for extra protection or absorbency. In various embodiments, a triple thick silver impregnated ABD dressing may be utilized.

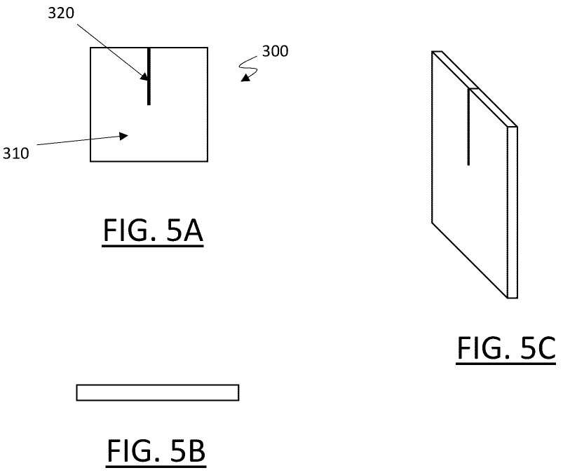

FIGS. 5A through 5C depict various views of a fixation or securement tab 500 which can be utilized with the various components described herein. Desirably, the securement tab 500 will comprise a tab body 510 formed from a flexible and/or deformable material, having at least one slot 520 formed therein. In use, the tab can accommodate a fixation pin or rod, with the slot sliding over the pin or rod and desirably retaining the pin or rod within the slot in a removeable manner.

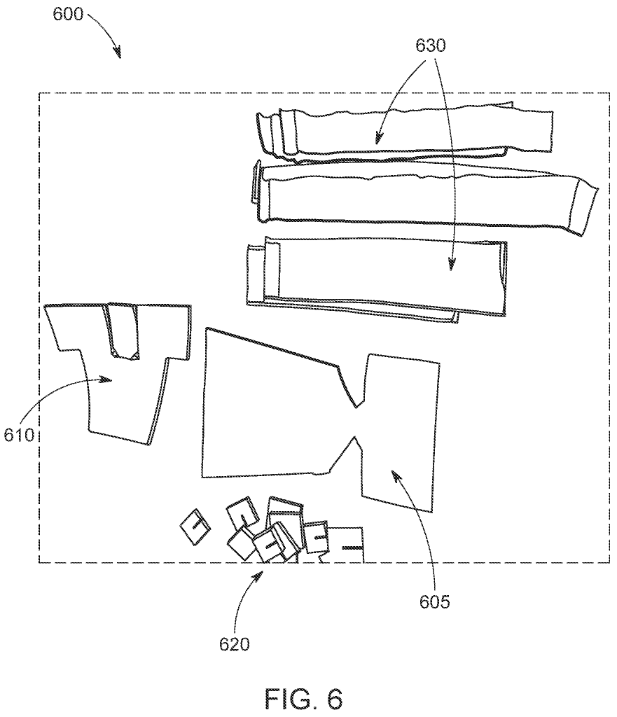

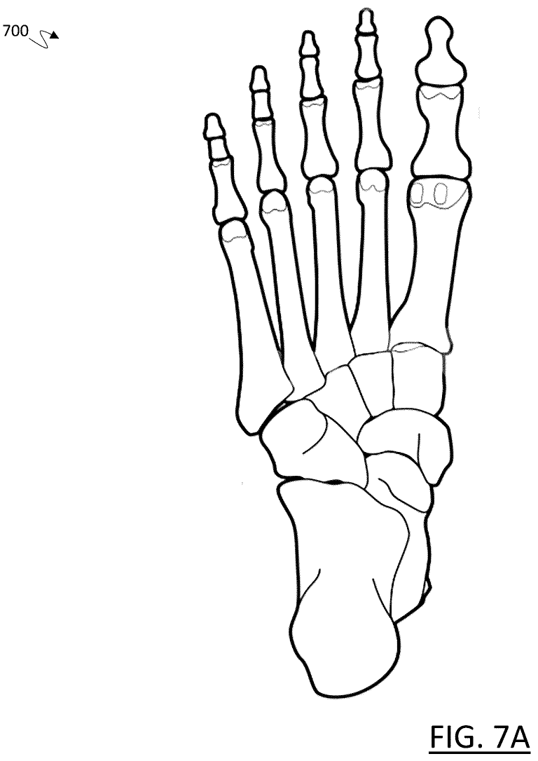

FIG. 6 depicts a plan view of one exemplary kit containing various of the wound dressing components described herein. In this embodiment, a first wound dressing component 605 and a second wound dressing component 610 are provided, along with a plurality of securement tabs 620. Also depicted are Ace strips 630 or gauze/adhesive strips, which may comprise and/or be secured using adhesive and/or self-securing features as well know to those of ordinary skill in the art.

In one exemplary embodiment, a dressing kit for a foot or other extremity secured within a circular fixation frame could include 2 to 4 ace strips 1×14 inches and 2 ace strips 2×14 inches, with attaching Velcro. The kit could also include 4-1 inch square and 4-2 inch square securement tabs to hold the various dressing components in place on the pins in the frame, until the dressing components can be secured in a desired position with the Ace bandage or other securement strips. If desired, the securement tabs could be utilized to hold gauze squares in place as well.

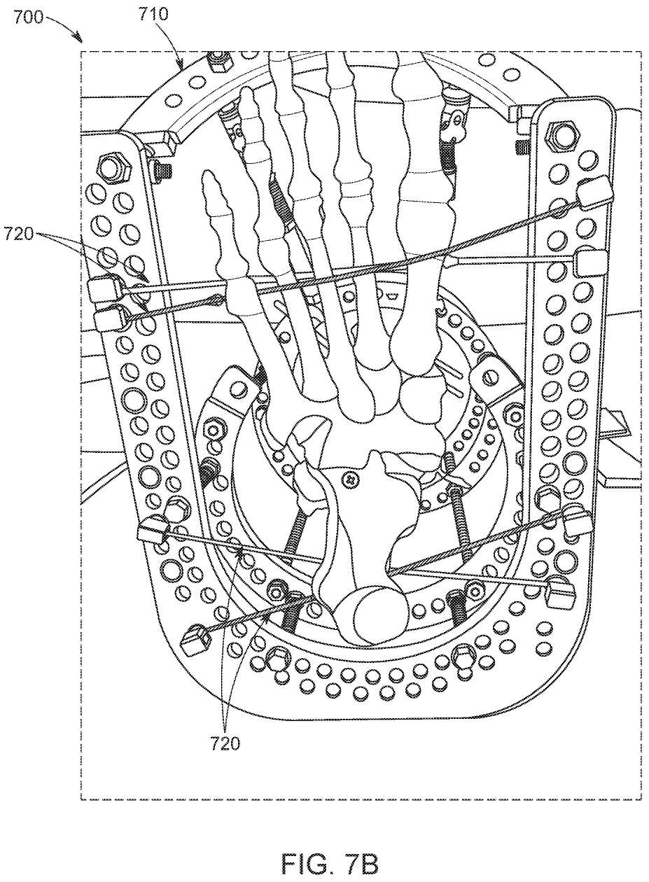

FIG. 7A and 7B depict an inferior view of the underside of a human skeletal foot 700, with the foot 700 depicted in FIG. 7B secured into a fixation frame 710 using pins 720.

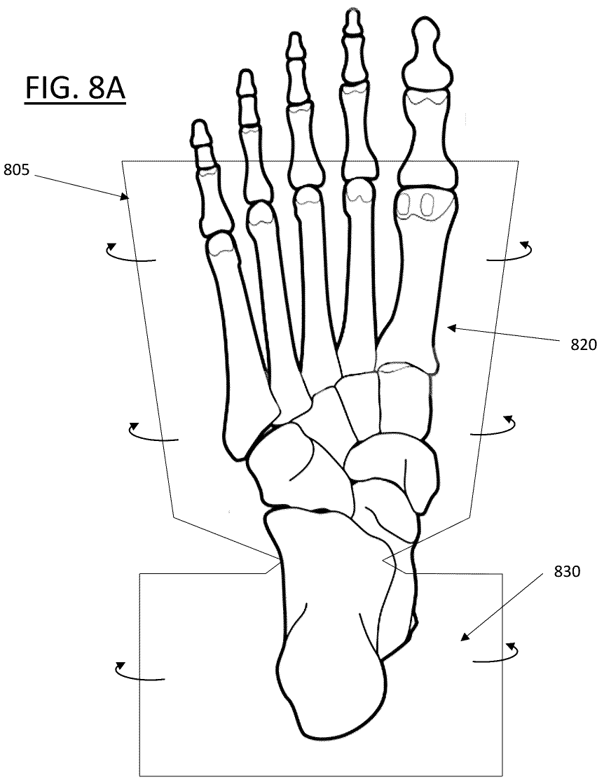

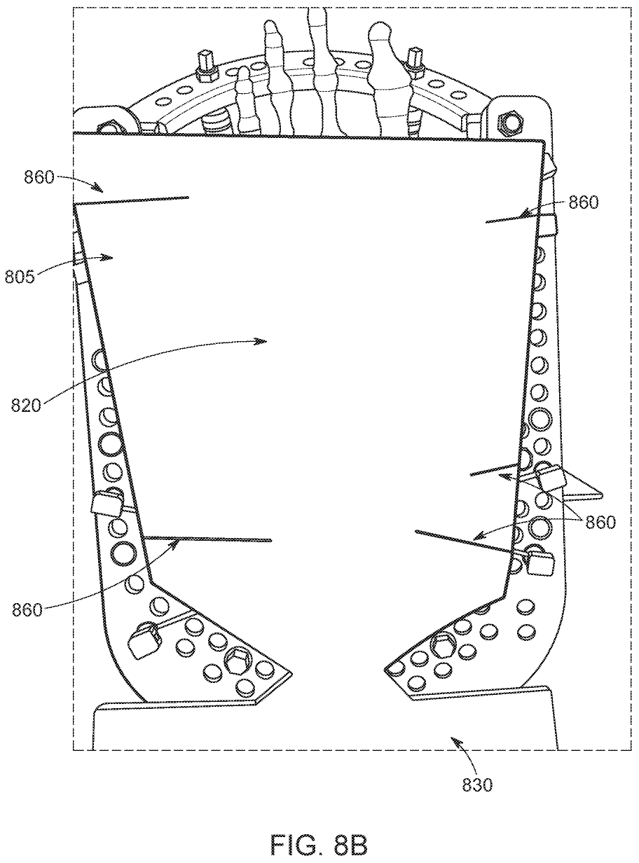

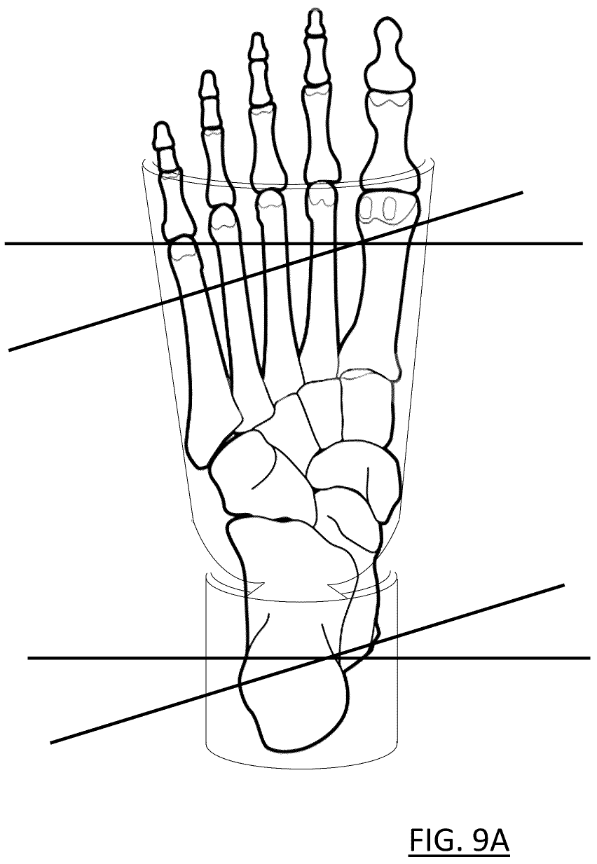

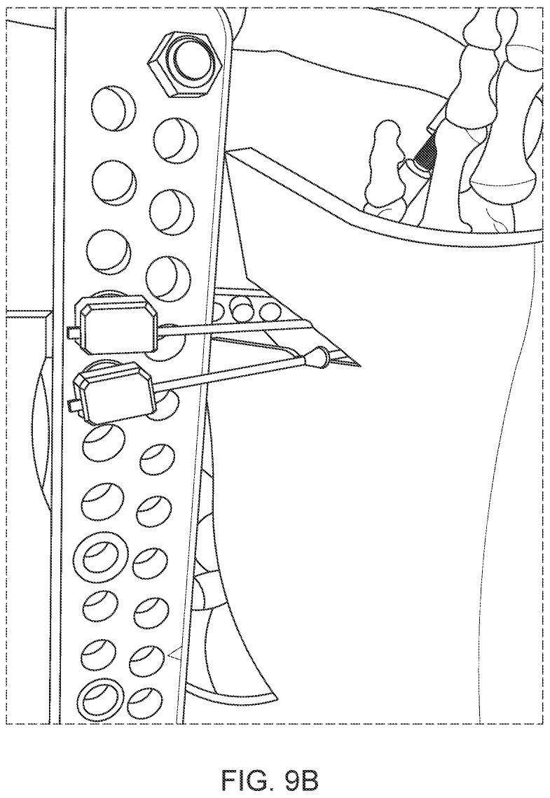

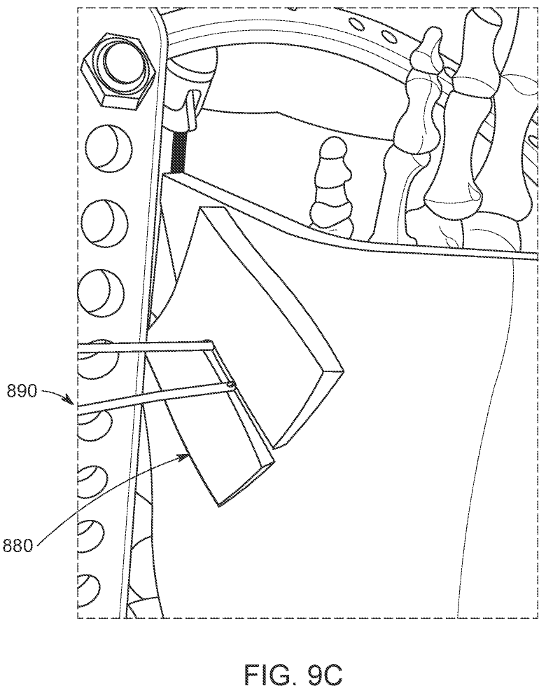

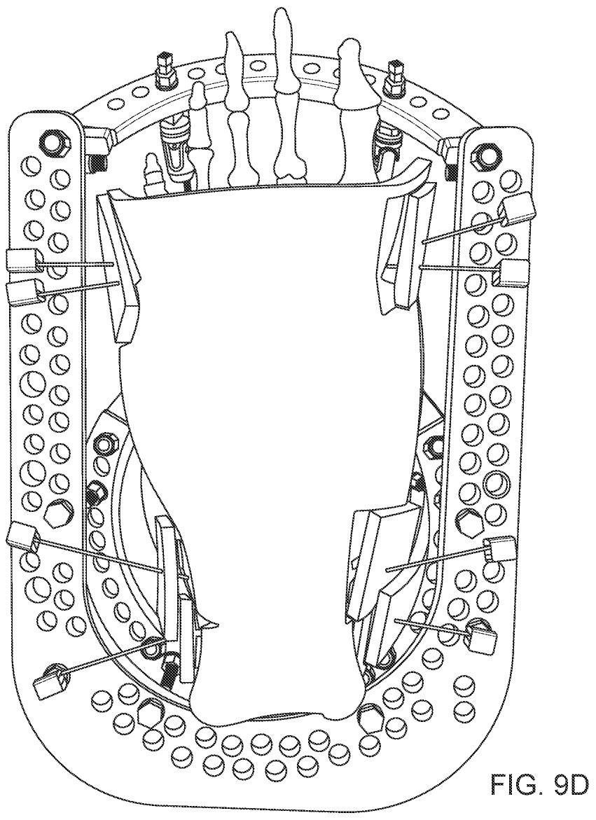

As best seen in FIGS. 8A and 8B, a first wound dressing component 805 can be positioned against the lower surface of the foot, with the central body 820 positioned against the arch of the foot and the base 830 positioned against the heel. The body tabs of the central body 820 are then wrapped around the foot, and the base tabs can be wrapped around the heel and sides of the ankle, as best shown in FIGS. 9A and 9B. If desired, the first wound dressing component 805 may include one or more slots 860, which can be pre-formed into the component 805, may be added by a user (e.g., using a cutting tool such as a scalpel or scissors), or the dressing component 805 may include pre-defined cut lines and/or may be tearable by the user to accommodate the pins for securement to the fixation pins. If desired, one or more securement tabs 880 can be attached to the fixation pins 890 (see FIGS. 9C and 9D) to temporarily and/or permanently hold the wound dressing component 805 in a desired position on the foot.

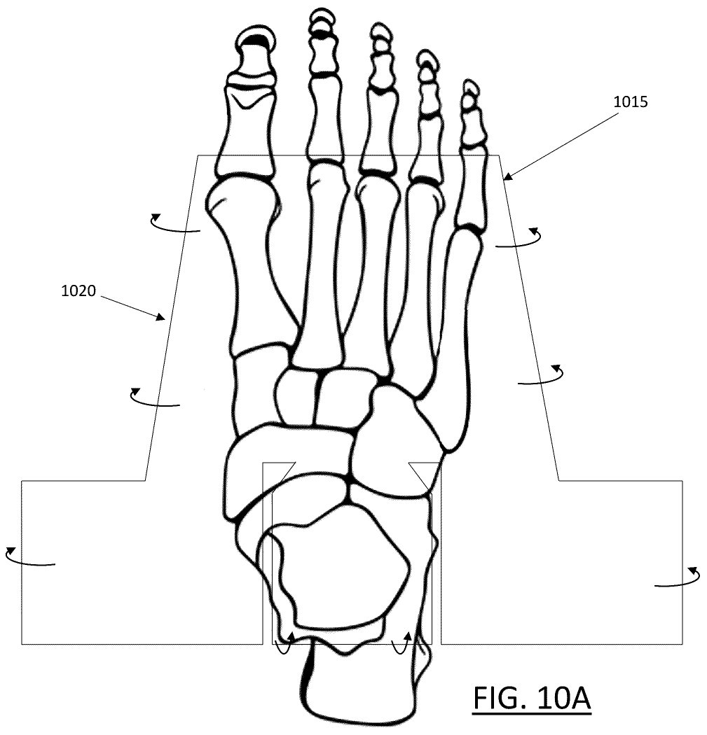

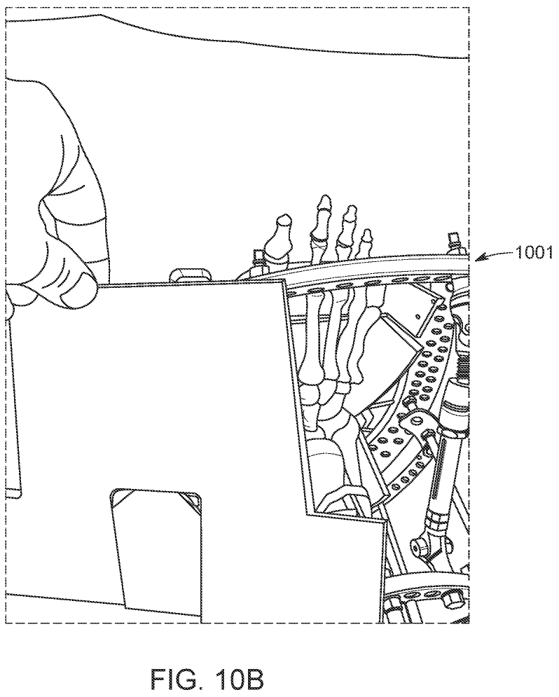

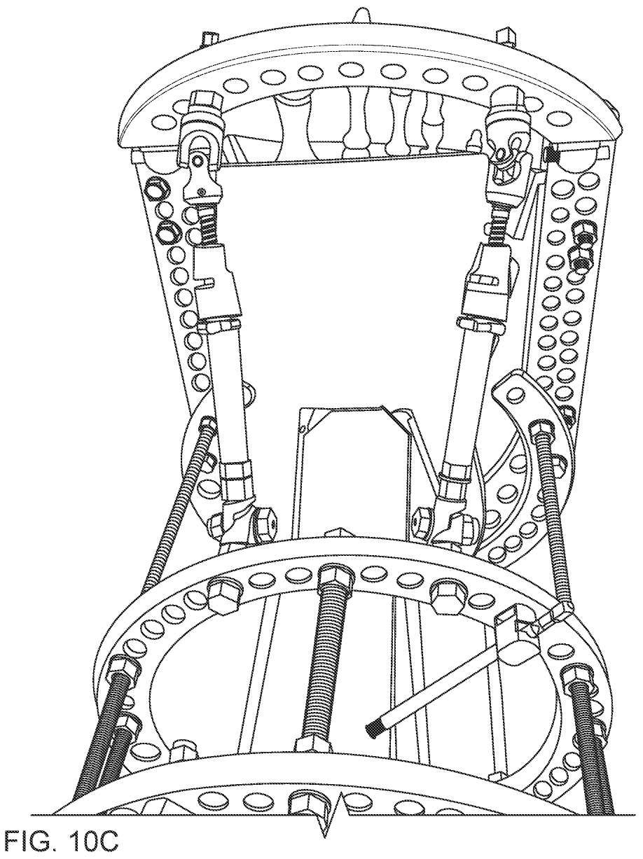

FIGS. 10A and 10B depict a superior view of the top of the human skeletal foot, with the foot depicted in FIG. 10B secured into a fixation frame 1001 using pins 1012. In this embodiment, a second wound dressing component 1015 can be positioned against the upper surface of the foot, with the central body 1020 positioned against the top of the foot, the center tab bent back and in contact with the patient's leg, and the tab sections wrapping around the sides to the patient's ankle (see FIG. 10C). If desired, one or more securement tabs can be attached to the fixation pins to temporarily and/or permanently hold the wound dressing component in a desired position on the foot.

If desired, the user may add extra gauze as needed, such as prior to application of the dressing kit, during dressing application and/or after. The user will desirably use securement tabs as necessary to secure and/or stabilize the dressing, and the 2-inch Ace strips (or other securement strips or other devices) as needed.

The disclosed embodiment will desirably at least partially protect the Achilles tendon, as well as the anterior ankle and skin. Of course, one of ordinary skill in the art should understand that the shape of the dressing components can be modified to add or subtract protection for various anatomical features of the foot, leg and/or ankle.

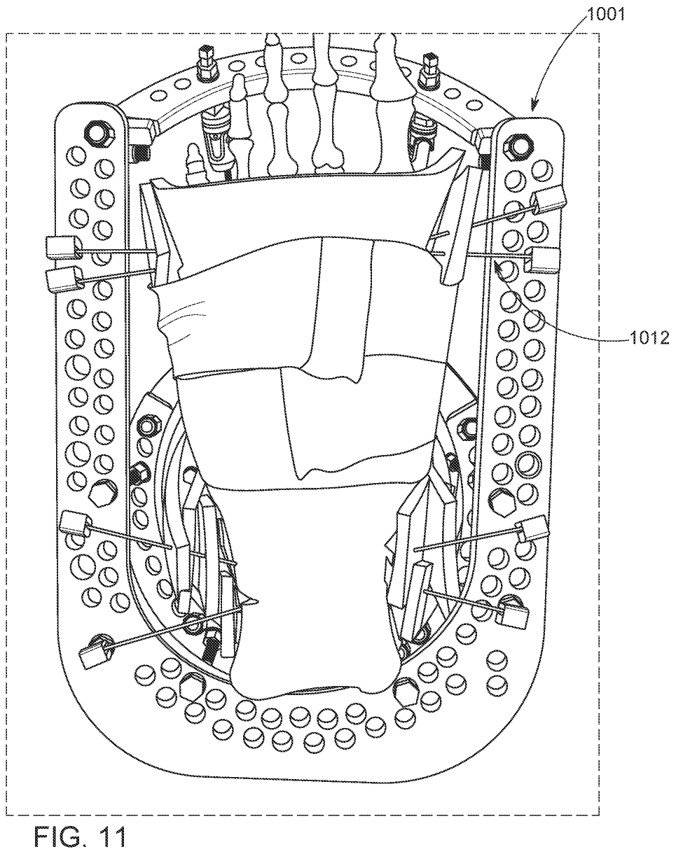

Once the first and second dressing portions are in a desired position, one or more Ace strips and/or gauze/adhesive strips may be wrapped around the dressing components and the foot, desirably securing the dressing components against the foot in a desired position and/or orientation, as depicted in FIG. 11.

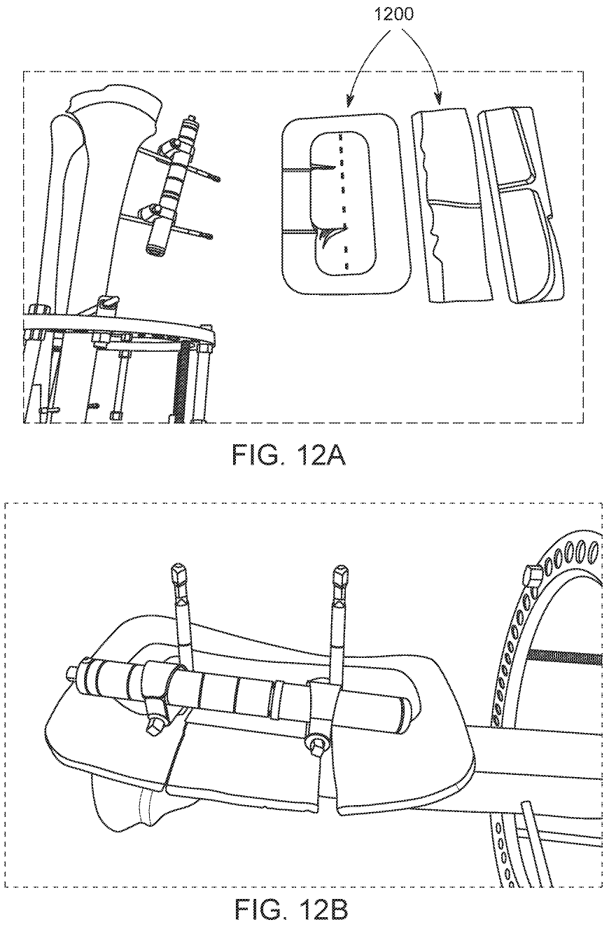

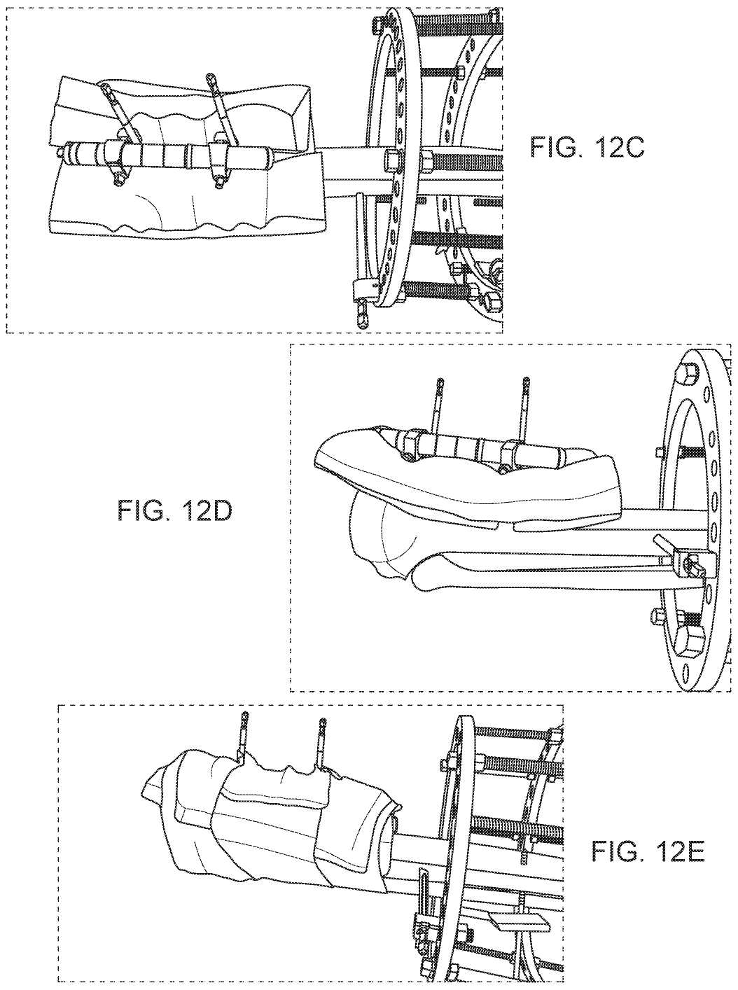

Another significant problem experienced with securement of an extremity in a fixation frame is the tendency of the extremity to swell up into the rail or other fixation frame components. Often, an end portion or surface of a fixation bar or other frame element can contact the extremity and cause a sore spot on the extremity. In various embodiments, a wound dressing component 1200 can be provided which can isolate and/or cushion a harder frame element from undesirable skin or other extremity contact. As best seen in FIG. 12A, a wound dressing component 1200 can comprise a generally rectangular sheet of flexible material such as a heavy-duty dressing material optionally capable of absorbing fluids, such as an abdominal gauze dressing or ABD pad, and/or optionally a foam bar.

As best seen in FIG. 12B, the wound dressing component can be secured to fixation pins or similar frame components via slits in the component. Alternatively, the component may comprise a foam block such as depicted in FIGS. 12C and 12D.

Once a desired component has been attached to the desired frame components, the user may add extra gauze as needed, such as prior to application of the dressing kit, during dressing application and/or after. The user will desirably use securement tabs as necessary to secure and/or stabilize the dressing, and the 2-inch Ace strips (or other securement strips or other devices) as needed (see FIG. 12E).

The various dressing components disclosed herein are easy to use and can be sold in a kit for a variety of extremities and/or surgical needs.

In various alternative embodiment, such as for circular frames without a foot plate, and exemplary dressing kit might include rectangular pads, square securement tabs to hold the dressing in place and ace strips with Velcro. An exemplary application of such components could be similar to the described application above, but in general the absence of the foot plate can make the application and securement of the dressing to the foot much easier.

Various disclosed embodiments can be for long bones and sized for the forearm and/or lower leg. For example, a small kit might be 20 cm×15 cm coverage, a medium kit might include 25 cm×20 cm coverage and a large kit could include 30 cm×25 cm of dressing component coverage. The enclosed Ace wrap strips and/or other securement devices could be similar for all kit sizes to reduce require inventory, such as 4 Ace strips of 2-inch width by 16 inches long, which size would desirably allow for overlap and extra padding used in free muscle flaps protected by circular framing.

In various embodiments, the pin to bar dressings can be sized according to the bar being used. The dressing can optionally be as long as the bar, and may include half that measurement for width. In general, the larger the bar, the thicker the below bar pad. If desired, the buttress next to the bar can be compressible (e.g., a foam pad) but it is also important to keep the swelling from pushing up onto and over the bar used. Exemplary dressing kits could come with several ace strips, such as a large dressing 1 strip having a 2 inch wide by 14-inch-long strip and two 1-inch-wide strips that are each 14 inches long. Such a set might not need the separate securement tabs used to hold the dressing as it does for the circular frame.

The various kits and components described herein a merely exemplary, and dressing sizes may vary according to patient size, weight, age, sex, and/or many other considerations. Dressings for children can be adjusted according to the sizes needed. The ratios of the dressings for children may be close to those used for adult size circular fixation with and without foot plates.

The dressing components may desirably be modular to allow adaptation to any position and/or injury location. In some embodiment that can be strips of compression wrap with Velcro to be easy to apply over the dressing and allow gentle compression (which may also allow the straps to be easily apply under the external fixation supports, if needed.) The dressing will desirably be easy to apply and remove in the operating room as well as an office setting.

While the disclosed devices and system can be useful in a variety of situations, the present invention may be particular useful during procedures employing orthopedic fixation systems, assemblies, devices and related methods for reduction of a fractured bone of a patient. While the disclosed embodiments are described in conjunction with a foot, it should be understood that the disclosed components may be equally applicable, with minor variations, to other extremities such as the hands, arms, feet, legs, head, etc.

The specific dimensions of any of the systems, assemblies, and components thereof, of the present invention can be readily varied depending upon the intended application, as will be apparent to those of skill in the art in view of the disclosure herein. Moreover, it is understood that the examples and embodiments described herein are for illustrative purposes only and that various modifications or changes in light thereof may be suggested to persons skilled in the art and are included within the spirit and purview of this application and scope of the appended claims. Numerous different combinations of embodiments described herein are possible, and such combinations are considered part of the present invention. In addition, all features discussed in connection with any one embodiment herein can be readily adapted for use in other embodiments herein. The use of different terms or reference numerals for similar features in different embodiments does not necessarily imply differences other than those which may be expressly set forth. Accordingly, the present invention is intended to be described solely by reference to the appended claims, and not limited to the preferred embodiments disclosed herein.

The entire disclosure of each of the publications, patent documents, and other references referred to herein is incorporated herein by reference in its entirety for all purposes to the same extent as if each individual source were individually denoted as being incorporated by reference.

### Equivalents

The invention may be embodied in other specific forms without departing from the spirit or essential characteristics thereof. The foregoing embodiments are therefore to be considered in all respects illustrative rather than limiting on the invention described herein. Scope of the invention is thus intended to include all changes that come within the meaning and range of equivalency of the descriptions provided herein.

### General

Many of the aspects and advantages of the present invention may be more clearly understood and appreciated by reference to the accompanying drawings. The accompanying drawings are incorporated herein and form a part of the specification, illustrating embodiments of the present invention and together with the description, disclose the principles of the invention.

Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the disclosure herein.

The various headings and titles used herein are for the convenience of the reader, and should not be construed to limit or constrain any of the features or disclosures thereunder to a specific embodiment or embodiments. It should be understood that various exemplary embodiments could incorporate numerous combinations of the various advantages and/or features described, all manner of combinations of which are contemplated and expressly incorporated hereunder.

The use of the terms “a” and “an” and “the” and similar referents in the context of describing the invention are to be construed to cover both the singular and the plural, unless otherwise indicated herein or clearly contradicted by context. The terms “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., i.e., “such as”) provided herein, is intended merely to better illuminate the invention and does not pose a limitation on the scope of the invention unless otherwise claimed. No language in the specification should be construed as indicating any non-claimed element as essential to the practice of the invention.

1. A dressing kit for treating an extremity attached to a fixation frame, comprising:
a first dressing component comprising an absorbent flexible dressing body having a plurality of attachment tabs, and
a second dressing component comprising an absorbent flexible dressing body having a plurality of attachment tabs, the first dressing component having a different configuration than the second dressing component,
wherein the first dressing component covers a first portion of the extremity but not a second portion of the extremity and the second dressing component covers the second portion of the extremity but not the first portion of the extremity and at least a first portion of the first dressing component overlaps at least a second portion of the second dressing component, and
wherein the first and second dressing components can be applied to the extremity attached to a fixation frame without requiring adjustment of an element of the fixation frame.

2. The dressing kit of claim 1, wherein the extremity attached to the fixation frame is a patient's foot.

3. The dressing kit of claim 1, wherein the extremity attached to the fixation frame is a patient's hand.
